(19) United States
(12) Patent Application Publication
(10) Pub. No.: US 2023/0368883 A1
(43) Pub. Date: Nov. 16, 2023

(54) SYSTEM AND METHODS FOR HARMONIZING AND ANALYZING MEDICAL DATA

(71) Applicant: Medalynx, Inc., Thousand Oaks, CA (US)

(72) Inventor: David Brown, Wiltshire (GB)

(73) Assignee: Medalynx, Inc., Thousand Oaks, CA (US)

(21) Appl. No.: 18/030,498

(22) PCT Filed: Oct. 1, 2021
(86) PCT No.: PCT/US2021/053257

§ 371 Date: Apr. 5, 2023

Related U.S. Application Data
(60) Provisional application No. 63/087,449, filed on Oct. 5, 2020.

(51) Int. Cl. G16H 20/10; G16H 10/60; G16H 50/70; G16H 40/20

(52) U.S. Cl. CPC G16H 20/10; G16H 10/60; G16H 50/70; G16H 40/20

(57) ABSTRACT

A medical data analysis system and associated methods are disclosed for automatically and dynamically collecting, harmonizing and analyzing medical data to identify and address abnormal prescribing behaviors. In at least one embodiment, upon a user desiring to obtain an analysis of a given medical condition, a model of expected prescribing behaviors for said medical condition is generated. Medical service providers stored within the system are stratified into a plurality of groups. A model of average prescribing behaviors for said medical condition is generated for each of the stratified groups of medical service providers. Upon determining that a given stratified group does not approximate the model of expected prescribing behaviors for said medical condition, the stratified group is identified as containing abnormal prescribing behaviors, and it is then determined which of the associated patient demographic and/or practice demographic data points had the strongest influence on the abnormal prescribing behaviors.

### RELATED APPLICATIONS

This application claims priority and is entitled to the filing date of U.S. provisional application Ser. No. 63/087,449, filed on Oct. 5, 2020. The contents of the aforementioned application are incorporated herein by reference.

### BACKGROUND

The subject of this provisional patent application relates generally to medical data, and more particularly to a system and associated methods for automatically and dynamically collecting, harmonizing and analyzing medical data to identify and address abnormal to prescribing behaviors.

Applicant(s) hereby incorporate herein by reference any and all patents and published patent applications cited or referred to in this application.

By way of background, genomic and clinical data from “real-world” sources provide insights needed for the more efficient development, clinical trial patient selection and prescribing of drugs used to treat diseases including cancer, cardiovascular, rare disease, autoimmune, neurological, diabetes and others and eventually help to obtain the individualized precision medicine future. Currently, most genomic, patient phenotypic and clinical data (hereinafter referred to generally as “patient data”) is sitting at academic and commercial sites around the world that are either not easily located or known. While the patient data at these same sites is ready to be accessed and analyzed, there remains a need for a system and associated methods for aggregating and harmonizing the patient data from disparate sources to allow for meaningful analysis across multiple, different patient data sources at scale.

Relatedly, with respect to use of such patient data to prescribe drugs and treatment regimens, while certain prescribing behaviors might be expected (based on various data points), it is sometimes the case that physicians aren't treating their patient base as expected (hereinafter referred to generally as “abnormal prescribing behavior”) for various reasons—including but not limited to clinical and socio-economic factors. Thus, as part of the necessary aggregation and harmonization of patient data, there also remains a need for a system and associated methods for identifying and addressing abnormal prescribing behaviors, so as to correct such abnormal prescribing behaviors and better ensure that such abnormal prescribing behaviors do not skew the analysis of patient data.

Aspects of the present invention fulfill these needs and provide further related advantages as described in the following summary.

It should be noted that the above background description includes information that may be useful in understanding aspects of the present invention. It is not an admission that any of the information provided herein is prior art or relevant to the presently claimed invention, or that any publication specifically or implicitly referenced is prior art.

### SUMMARY

Aspects of the present invention teach certain benefits in construction and use which give rise to the exemplary advantages described below.

The present invention solves the problems described above by providing a medical data analysis system and associated methods for automatically and dynamically collecting, harmonizing and analyzing medical data to identify and address abnormal prescribing behaviors. In at least one embodiment, upon a user desiring to obtain an analysis of a given medical condition, a model of expected prescribing behaviors for said medical condition is generated, organized by an at least one pharmaceutical product being prescribed for treating said medical condition, based on existing and generally accepted clinical guidelines. Medical service providers stored within the system are stratified into a plurality of groups based on at least one of the respective patient demographic data points of the associated medical service providers and the prescription performance indicator of each associated patient that has been treated, or is being treated, by each of the medical service providers. A model of average prescribing behaviors for said medical condition, organized by the at least one pharmaceutical product being prescribed for treating said medical condition, is generated for each of the stratified groups of medical service providers. Upon determining that a given stratified group does not approximate the model of expected prescribing behaviors for said medical condition, the stratified group is identified as containing abnormal prescribing behaviors, and it is then determined which of the associated patient demographic and/or practice demographic data points had the strongest influence on the abnormal prescribing behaviors for the associated medical service providers so that appropriate corrective actions may be taken.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

The accompanying drawings illustrate aspects of the present invention. In such drawings:

FIG. 1 is a simplified schematic view of an exemplary medical data analysis system, in accordance with at least one embodiment;

FIG. 2 is an architecture diagram of an exemplary patient record, in accordance with at least one embodiment;

FIG. 3 is an architecture diagram of an exemplary service provider record, in accordance with at least one embodiment;

FIG. 4 is a flow diagram of an exemplary method of collecting and harmonizing medical data, in accordance with at least one embodiment;

FIG. 5 is a flow diagram of an exemplary method of analyzing medical data to identify and address abnormal prescribing behaviors, in accordance with at least one embodiment; and

FIG. 6 is a diagram of a line graph illustrating stratified prescribing behaviors as compared to an expected prescribing behavior for treating a given medical condition, in accordance with at least one embodiment.

The above described drawing figures illustrate aspects of the invention in at least one of its exemplary embodiments, which are further defined in detail in the following description. Features, elements, and aspects of the invention that are referenced by the same numerals in different figures represent the same, equivalent, or similar features, elements, or aspects, in accordance with one or more embodiments.

### DETAILED DESCRIPTION

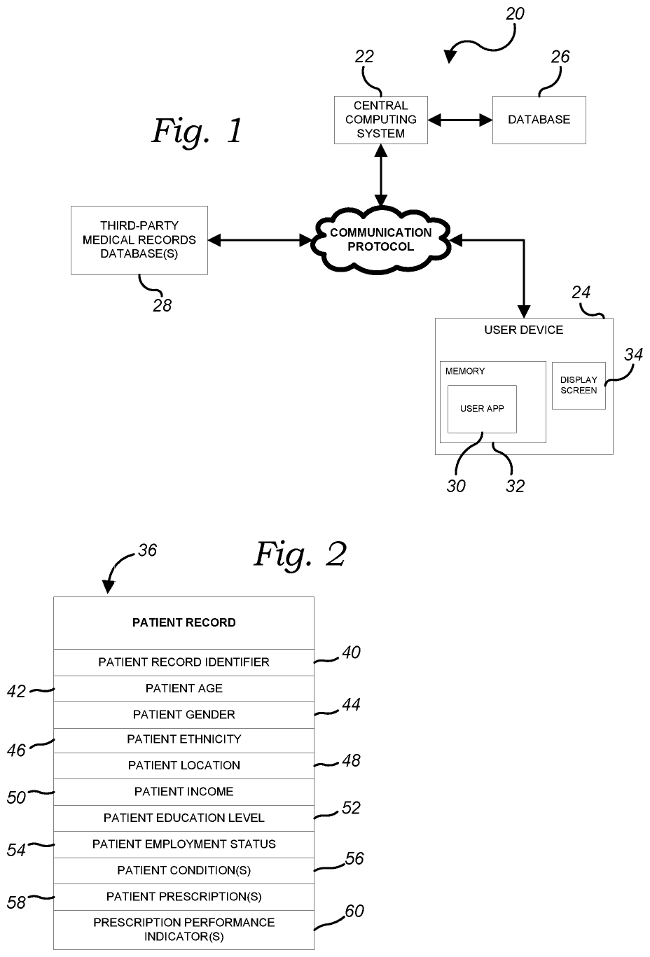

Turning now to FIG. 1, there is shown a simplified schematic view of an exemplary medical data analysis system 20 configured for automatically and dynamically collecting, harmonizing and analyzing medical data to identify and address abnormal prescribing behaviors, in accordance with at least one embodiment. In at least one embodiment, the system 20 provides a central computing system 22 configured for receiving and processing select data related to an at least one patient and an associated at least one medical condition, along with an at least one medical service provider tasked with treating the at least one patient. In that regard, it should be noted that the term “medical service provider” is intended to include (but is in no way limited to) physicians, nurses, clinicians, hospitals, clinics and any other type of medical professional or medical entity who may provide medical services to the at least one patient. In at least one embodiment, an at least one user device 24 is in selective communication with the computing system 22, as discussed further below. Additionally, in at least one embodiment, an at least one database 26 is in communication with the computing system 22 and configured for selectively storing said data related to each of the at least one patient, at least one medical condition, and at least one medical service provider. In at least one embodiment, the computing system 22 and database 26 are one to and the same—as such, it is intended that those terms as used herein are to be interchangeable with one another. In at least one embodiment, the computing system 22 and database 26 are omitted, such that the system 20 and associated methods described herein are implemented solely through the at least one user device 24—thus, any methods or functionality described herein as being carried out by the computing system 22 or database 26 may, in at least one embodiment, also be carried out by the at least one user device 24, regardless of whether such embodiments nevertheless incorporate the computing system 22 and/or database 26. In at least one embodiment, the computing system 22 is also in selective communication with an at least one third-party medical records database 28 (public and/or private) containing data related to the at least one patient, at least one medical condition, and/or at least one medical service provider, as discussed further below.

At the outset, it should be noted that communication between each of the computing system 22, at least one user device 24, at least one database 26, and at least one medical records database 28 may be achieved using any wired- or wireless-based communication protocol (or combination of protocols) now known or later developed. As such, the present invention should not be read as being limited to any one particular type of communication protocol, even though certain exemplary protocols may be mentioned herein for illustrative purposes. Similarly, in at least one embodiment, communications between each of the computing system 22, at least one user device 24, at least one database 26, and at least one medical records database 28 may be encrypted using any encryption method (or combination of methods) now known or later developed. It should also be noted that the term “user device” is intended to include any type of computing or electronic device, now known or later developed, capable of communicating with the computing system 22 and carrying out the functionality described herein—such as desktop computers, browser extensions, mobile phones, smartphones, laptop computers, tablet computers, personal data assistants, gaming devices, wearable devices, etc. As such, the present invention should not be read as being limited to use with any one particular type of computing or electronic device, even though certain exemplary devices may be mentioned or shown herein for illustrative purposes.

With continued reference to FIG. 1, in the exemplary embodiment, each of the computing system 22, at least one user device 24, and at least one database 26 contains the hardware and software necessary to carry out the exemplary methods for collecting, harmonizing and analyzing medical data, as described herein. Furthermore, in at least one embodiment, the computing system 22 comprises a plurality of computing devices selectively working in concert with one another to carry out the exemplary methods for administering the medical data analysis system 20, as described herein. In at least one to embodiment, the at least one user device 24 provides a user application 30 residing locally in memory 32 on the user device 24 (either as a standalone application or as a browser extension for an existing Internet browser on the user device 24), the user application 30 being configured for selectively communicating with the computing system 22, as discussed further below. It should be noted that the term “memory” is intended to include any type of electronic storage medium (or combination of storage mediums) now known or later developed, such as local hard drives, RAM, flash memory, secure digital (“SD”) cards, external storage devices, network or cloud storage devices, integrated circuits, etc. Additionally, in at least one embodiment, each of the at least one user device 24 is in the possession of a user who is desirous of utilizing the system 20 to automatically identify and address abnormal prescribing behaviors.

Furthermore, the various components of the at least one user device 24 may reside on a single computing and/or electronic device, or may separately reside on two or more computing and/or electronic devices in communication with one another. In at least one alternate embodiment, the functionality provided by the user application 30 resides remotely in memory on the computing system 22 and/or database 26, with the at least one user device 24 capable of accessing said functionality via an online portal hosted by (or at least in communication with) the computing system 22 and/or database 26, either in addition to or in lieu of the user application 30 residing locally in memory 32 on the at least one user device 24. It should be noted that, for simplicity purposes, the functionality provided by the user application 30 and/or computing system 22 will be described herein as such—even though certain embodiments may provide said functionality through an online portal. It should also be noted that, for simplicity purposes, when discussing functionality and the various methods that may be carried out by the system 20 herein, the terms “user device” and “user application” are intended to be interchangeable. With continued reference to FIG. 1, in at least one embodiment, the at least one user device 24 provides an at least one display screen 34 for providing an at least one graphical user interface to assist the associated user in possession of said user device 24 to access and utilize the various functions provided by the system 20.

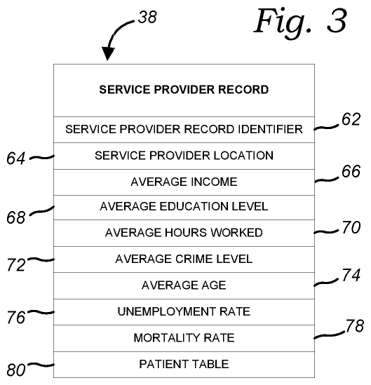

In at least one embodiment, as illustrated in the architecture diagrams of FIGS. 2 and 3, the computing system 22 and/or the at least one database 26 stores and manages a patient record 36 (FIG. 2) associated with each of the at least one patient (containing various details related to said at least one patient), and a service provider record 38 (FIG. 3) associated with each of the at least one medical service provider (containing various details related to said at least one medical service provider).

In at least one embodiment, each patient record 36 contains at least one of a unique patient record identifier 40, a patient age 42, a patient gender 44, a patient ethnicity 46, a patient location 48, a patient income 50, a patient education level 52, a patient employment status 54, an at least one patient condition 56 for each medical condition the associated patient has experienced or is experiencing, an associated patient prescription 58 for each of the at least one patient condition 56, and an associated at least one prescription performance indicator 60 for each of the at least one patient prescription 58. For example, where the patient condition 56 is Type I diabetes, the associated patient prescription 58 might be 10 units of short-acting insulin before each meal, and the associated prescription performance indicator 60 might be the patient's hemoglobin A1c (“HbA1c”) level. In further embodiments, additional patient- and/or general demographic-related data points, now known or later developed, may be collected, harmonized and analyzed by the system 20 to carry out the methods described herein. In at least one embodiment, the at least one patient record 36 contains no personally identifiable information that would allow for a given patient record 36 to be linked to a specific individual.

In at least one embodiment, each service provider record 38 contains at least one of a unique service provider record identifier 62, a service provider location 64, an average income 66 representing the average income of patients treated by the associated medical service provider, an average education level 68 representing the average education level of patients treated by the associated medical service provider, an average hours worked 70 representing the average hours worked by patients treated by the associated medical service provider, an average crime level 72 representing the average crime level in the corresponding service provider location 64, an average age 74 representing the average age of patients treated by the associated medical service provider, an unemployment rate 76 representing the unemployment rate in the corresponding service provider location 64, a mortality rate 78 representing the mortality rate in the corresponding service provider location 64, and a patient table 80 containing links to the corresponding patient record 36 of each patient that has been treated by the associated medical service provider. With each service provider record 38 containing the associated patient table 80, the system 20 is capable of calculating certain statistics for a given medical service provider, such as a total quantity of patients being treated by a given medical service provider for a given medical condition, or an average age of patients being treated by a given medical service provider for a given medical condition—or even more specific statistics, such as a total quantity of Hispanic female patients between the ages of 18 and 35 being treated by a given medical service provider for a given medical condition. In further embodiments, additional practice-related data points, now known or later developed, may be collected, harmonized and analyzed by the system 20 to carry out the methods described herein. It should also be noted that while the term “table” is used herein to describe certain exemplary data structures, in at least one embodiment, any other suitable data type or data structure, or combinations thereof, now known or later developed, capable of storing the appropriate data, may be substituted. Thus, the present invention should not be read as being so limited.

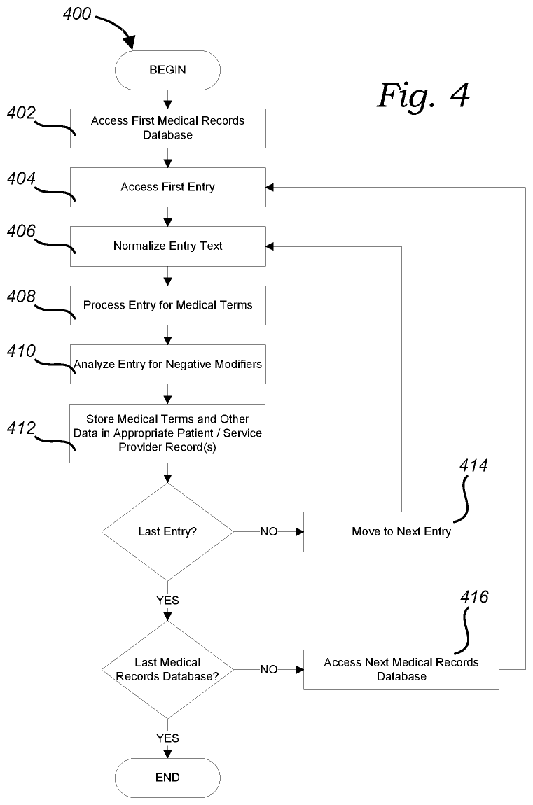

Before the system 20 is able to analyze medical data, that medical data must first be collected and harmonized. A major barrier in the analysis of real-world evidence derived from electronic medical records is the availability of patient data in a structured, standardized form. Most of the important patient data held in the third-party medical records databases 28 is contained in the form of medical notes entered by the medical service providers. Accordingly, the system 20 provides methods for automatically collecting and harmonizing the patient data. In at least one embodiment, as illustrated in the flow diagram of FIG. 4, through the user application 30 residing either locally in memory 32 on the at least one user device 24 or remotely on the computing system 22 and/or database 26, the computing system 22 first accesses each of the at least one third-party medical records database 28 (402) and retrieves one entry from said medical records database 28 at a time (404). For each entry, the computing system 22 normalizes the text of the entry (406)—such as making the entire string of text lowercase, for example—and processes the entry to identify any medical terms contained therewithin (408). In at least one such embodiment, the computing system 22 identifies medical terms by cross-referencing each word in the entry against one or more databases of medical terms. For each medical term identified, the computing system 22 examines the words surrounding the medical term to determine the presence or absence of negative modifiers (i.e., “no,” “does not,” etc.) that would affect whether the entry deals with the presence or absence of the identified medical term (410). In at least one embodiment, computing system 22 links the medical term with the proper codes associated with one or more medical encoding systems (such as ICD-10, OPCS, SNOMED CT, MEDRA, etc.). The medical term details, along with any other details related to the associated patient and/or medical service provider are saved into an appropriate patient record 36 and/or service provider record 38 within the system 20 (412). This process is repeated for each entry in each of the at least one third-party medical records database 28 (414, 416), and the at least one third-party medical records database 28 is periodically checked for any new entries to ensure the medical data in the system 20 is up to date.

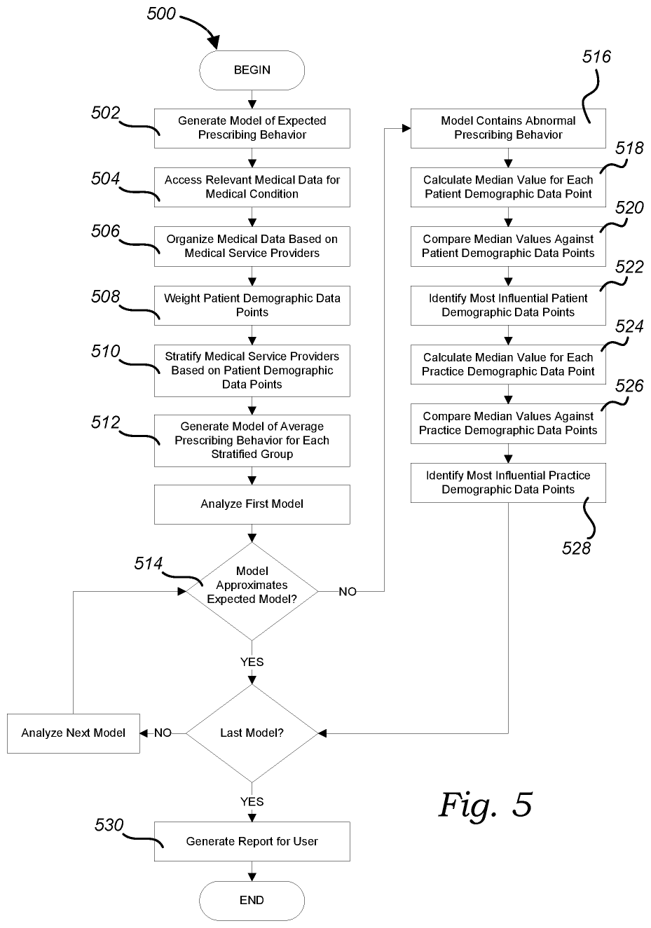

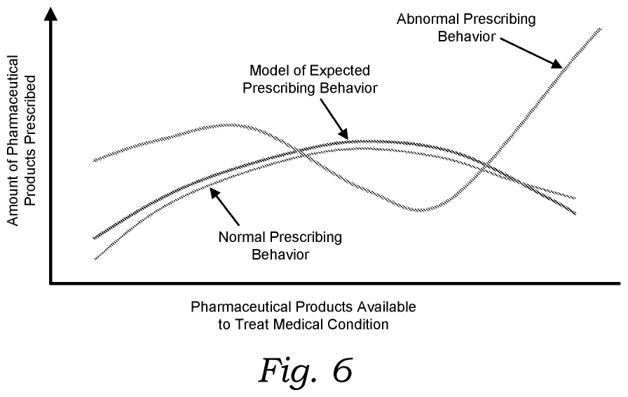

In at least one embodiment, as illustrated in the flow diagram of FIG. 5, upon a user desiring to utilize the system 20 to analyze a given medical condition, the computing system 22 first generates a model of an expected prescribing behavior for the medical condition, organized by the pharmaceutical products being prescribed for treating the medical condition, based on existing and generally accepted clinical guidelines (502), as plotted on the line graph of FIG. 6 for illustrative purposes. The computing system 22 then accesses the collected and harmonized medical data related the medical condition (504) and organizes the medical data based on the associated medical service providers (506). In at least one such embodiment, the computing system 22 stratifies the medical service providers into a plurality of groups (510) based on at least one of their respective patient demographics (i.e., one or more of the demographic-related data points noted above in connection with the medical data stored in each service provider record 38) or the prescription performance indicator 60 of each associated patient that has been treated (or is being treated) by each of the medical service providers. By way of non-limiting example, in at least on embodiment, the medical service providers may be stratified based on one or more of: the total number of patients in the system 20; the respective ages of the patients in the system 20; the respective genders of the patients in the system 20; the respective ethnicities of the patients in the system 20; the total number of patients in the system 20 having the medical condition; the respective ages of the patients in the system 20 having the medical condition; the respective genders of the patients in the system 20 having the medical condition; the respective ethnicities of the patients in the system 20 having the medical condition; and the respective prescription performance indicator 60 associated with each of the patients in the system 20 having the medical condition (i.e., the achievement of specifically defined clinical markers within the patients registered with the medical condition—e.g., where the medical condition is Type I diabetes, the prescription performance indicator 60 may be the respective HbA1c level). It should be noted that, in at least one embodiment, the use of ethnicity of the patient population is used to identify any genetic propensity of the patient population to suffer from specific cardio-metabolic chronic diseases. As more direct patient genetic data becomes available the presence in the population of specific disease related genetic mutations will become another factor in stratification, in at least one embodiment.

In at least one embodiment, depending on the data points utilized by the computing system 22 to stratify the medical service providers, the computing system 22 weights the patient demographic data points based on the relative strength of their potential influence on prescribing behaviors (508)—i.e., based on their relative importance. In at least one such embodiment, patient demographic data points related to the medical condition being analyzed are accorded a relatively larger numerical weight than general patient demographic data points (i.e., not related to the medical condition), and the at least one prescription to performance indicator 60 associated with each of the patients in the system 20 having the medical condition is accorded a relatively larger numerical weight than other patient demographic data points related to the medical condition being analyzed. By way of non-limiting example, in at least one such embodiment, where the numerical weights range between 1 and 10, the total number of patients in the system 20 may be given a weight value of 4; the respective ages of the patients in the system 20 may be given a weight value of 5; the respective genders of the patients in the system 20 may be given a weight value of 3; the respective ethnicities of the patients in the system 20 may be given a weight value of 5; the respective ages of the patients in the system 20 having the medical condition may be given a weight value of 8; the respective genders of the patients in the system 20 having the medical condition may be given a weight value of 8; the respective ethnicities of the patients in the system 20 having the medical condition may be given a weight value of 9; and the respective prescription performance indicator 60 associated with each of the patients in the system 20 having the medical condition may be given a weight value of 10. It should be noted that the above-mentioned weight values are merely exemplary and intended to simply illustrate the exemplary method described herein. In further embodiments, other weight values may be utilized, so long as patient demographic data points related to the medical condition being analyzed are accorded a relatively larger numerical weight than general patient demographic data points, and the at least one prescription performance indicator 60 associated with each of the patients in the system 20 having the medical condition is accorded a relatively larger numerical weight than other patient demographic data points related to the medical condition being analyzed.

In at least one embodiment, once the computing system 22 stratifies the medical service providers into a plurality of groups (510), the computing system 22 generates a model of the average prescribing behaviors for the medical condition, organized by the pharmaceutical products being prescribed for treating the medical condition, for each of the stratified groups of medical service providers (512), as also plotted on the line graph of FIG. 6 for illustrative purposes. In at least one such embodiment, the model for each stratified group is based on the composite prescribing behaviors by the associated medical service providers who's patients fall into the defined groups. For simplicity purposes, the line graph of FIG. 6 only illustrates two stratified groups of medical service providers—one of which approximates the model of expected prescribing behaviors for the medical condition, and the other does not. In at least one embodiment, abnormal prescribing behaviors are determined by totaling the annual prescribing of each pharmaceutical product being prescribed for treating the medical condition (adjusted for the number of patients, suffering from the to medical condition, treated by the medical service provider) and comparing the prescribing behavior of a given medical service provider to the average in their stratified group. Upon the computing system 22 determining that a given stratified group does not approximate the model of expected prescribing behaviors for the medical condition (514), the computing system 22 identifies said stratified group as containing abnormal prescribing behaviors (516).

In at least one embodiment, for any stratified groups identified by the computing system 22 as containing abnormal prescribing behaviors, the computing system 22 determines which patient demographic data points (i.e., socioeconomic, non-medical factors) had the strongest influence on the abnormal prescribing behaviors for the associated medical service providers. In at least one embodiment, the computing system 22 first calculates the median value for each patient demographic data point that was used to generate the models for stratified groups (518). The computing system 22 then compares the calculated median values against the corresponding values for each patient demographic data point associated with each medical service provider identified as engaging in abnormal prescribing behaviors, in order to determine which of the patient demographic data points for a given medical service provider fail to track with the corresponding median values (520). This allows the computing system 22 to identify the strongest patient demographic data points related to the underlying causes of abnormal prescribing behaviors (522). In at least one such embodiment, the patient demographic data points for a given medical service provider that have the highest variance from the corresponding median values are identified as being the strongest factors in the abnormal prescribing behaviors.

In at least one embodiment, for any stratified groups identified by the computing system 22 as containing abnormal prescribing behaviors, the computing system 22 next determines whether any practice demographic data points (i.e., quantity of salaried physicians/nurses/clinicians employed by the medical service provider, quantity of temporary physicians/nurses/clinicians employed by the medical service provider, level of experience for each physician/nurse/clinician employed by the medical service provider, age of each physician/nurse/clinician employed by the medical service provider, gender of each physician/nurse/clinician employed by the medical service provider, ethnicity of each physician/nurse/clinician employed by the medical service provider, etc.) had an influence on the abnormal prescribing behaviors for the associated medical service providers. In at least one such embodiment, the various practice demographic data points are stored in the service provider record 38 associated with each medical service provider. In at least one to embodiment, the computing system 22 first calculates the median value for each practice demographic data point (524). The computing system 22 then compares the calculated median values against the corresponding values for each practice demographic data point associated with each medical service provider identified as engaging in abnormal prescribing behaviors, in order to determine which of the practice demographic data points for a given medical service provider (if any) fail to track with the corresponding median values (526). This allows the computing system 22 to identify the strongest practice demographic data points related to the underlying causes of abnormal prescribing behaviors (528). In at least one such embodiment, the practice demographic data points for a given medical service provider that have the highest variance from the corresponding median values are identified as being the strongest factors in the abnormal prescribing behaviors.

In at least one embodiment, upon the computing system 22 determining the patient and/or practice demographic data points having the strongest influence on the identified abnormal prescribing behaviors for a given medical service provider, the computing system generates a report for the user outlining said data points (530)—namely, providing information on the level of abnormal prescribing and identifying the data points having the strongest influence on the abnormal prescribing. In at least one embodiment, the report also contains recommendations as to how to counteract the influential data points in order to reduce or eliminate the abnormal prescribing behaviors. In at least one such embodiment, the types of recommendations are dependent upon the role of the user with the identified medical service provider. For example, the recommendations for a pharmaceuticals company will vary from those identified for a health care system based on the organization's ability to influence the identified data points.

Aspects of the present specification may also be described as the following embodiments:
1. A method for analyzing medical data to identify and address abnormal prescribing behaviors, the method comprising the steps of: implementing a central computing system in selective communication with an at least one third-party medical records database, the computing system configured for receiving and processing data related to an at least one patient and an associated at least one medical condition, along with an at least one medical service provider tasked with treating the at least one patient; the computing system establishing an at least one patient record associated with each of the at least one patient, each patient record containing at least one of a unique patient record identifier, a patient age, a patient gender, a patient ethnicity, a patient location, a patient income, a patient education level, a patient employment status, an at least one patient condition for each medical condition the associated patient has experienced or is experiencing, an associated patient prescription for each of the at least one patient condition, and an associated at least one prescription performance indicator for each of the at least one patient prescription; the computing system establishing an at least one service provider record associated with each of the at least one medical service provider, each service provider record containing at least one of a unique service provider record identifier, a service provider location, an average income representing the average income of patients treated by the associated medical service provider, an average education level representing the average education level of patients treated by the associated medical service provider, an average hours worked representing the average hours worked by patients treated by the associated medical service provider, an average crime level representing the average crime level in the corresponding service provider location, an average age representing the average age of patients treated by the associated medical service provider, an unemployment rate representing the unemployment rate in the corresponding service provider location, a mortality rate representing the mortality rate in the corresponding service provider location, and a patient table containing links to the corresponding patient record of each patient that has been treated by the associated medical service provider; and upon a user desiring to obtain an analysis of a given medical condition: the computing system generating a model of expected prescribing behaviors for said medical condition, organized by an at least one pharmaceutical product being prescribed for treating said medical condition, based on existing and generally accepted clinical guidelines; the computing system accessing data contained in the at least one service provider record related to said medical condition; the computing system stratifying the associated medical service providers into a plurality of groups based on at least one of the respective patient demographic data points of the associated medical service providers and the prescription performance indicator of each associated patient that has been treated, or is being treated, by each of the medical service providers; the computing system generating a model of average prescribing behaviors for said medical condition, organized by the at least one pharmaceutical product being prescribed for treating said medical condition, for each of the stratified groups of medical service providers; upon the computing system determining that a given stratified group does not approximate the model of expected prescribing behaviors for said medical condition, the computing system identifying said stratified group as containing abnormal prescribing behaviors; and for any stratified groups identified by the computing system as containing abnormal prescribing behaviors: the computing system determining which of the associated at least one patient demographic data point had the strongest influence on the abnormal prescribing behaviors for the associated medical service providers; and the computing system determining whether any practice demographic data points had an influence on the abnormal prescribing behaviors for the associated medical service providers.
2. The method according to embodiment 1, further comprising the step of implementing an at least one database in communication with the computing system and configured for selectively storing said data related to the at least one patient, medical condition and medical service provider.
3. The method according to embodiments 1-2, further comprising the steps of: the computing system accessing each of the at least one third-party medical records database and retrieving each of the entries contained within said medical records database; and for each entry retrieved from said medical records database: the computing system normalizing any text contained in said entry; the computing system processing said entry to identify any medical terms contained therewithin; for each medical term identified, the computing system examining any words surrounding said medical term to determine the presence or absence of negative modifiers that would affect whether said entry deals with the presence or absence of said medical term; and the computing system storing the data contained in said entry in at least one of a patient record and service provider record.
4. The method according to embodiments 1-3, further comprising the step of, for each medical term identified, the computing system linking said medical term with an at least one code associated with a standard medical encoding system.
5. The method according to embodiments 1-4, wherein the step of the computing system stratifying the associated medical service providers into a plurality of groups further comprises the step of the computing system weighting the associated at least one patient demographic data point based on the relative strength of said patient demographic data point's potential influence on prescribing behaviors.
6. The method according to embodiments 1-5, wherein the step of the computing system weighting the associated at least one patient demographic data point further comprises the steps of: the computing system assigning a relatively larger numerical weight to patient demographic data points related to the medical condition being analyzed than the numerical weight assigned to general patient demographic data points; and the computing system assigning a relatively larger numerical weight to the at least one prescription performance indicator associated with each of the at least one patient record having the medical condition than the numerical weight assigned to patient demographic data points related to the medical condition being analyzed.
7. The method according to embodiments 1-6, wherein the step of the computing system determining that a given stratified group does not approximate the model of expected prescribing behaviors for said medical condition, further comprises the steps of: the computing system totaling an annual prescribing amount of each pharmaceutical product being prescribed for treating said medical condition; and the computing system comparing the prescribing behavior of a given medical service provider to the average in the associated stratified group in which said medical service provider is categorized.
8. The method according to embodiments 1-7, wherein the step of the computing system determining which of the associated at least one patient demographic data point had the strongest influence on the abnormal prescribing behaviors for the associated medical service providers further comprises the steps of: the computing system calculating a median value for each patient demographic data point that was used to generate the model of average prescribing behaviors for said medical condition for each of the stratified groups; and the computing system comparing the calculated median values against the corresponding values for each patient demographic data point associated with each medical service provider identified as engaging in abnormal prescribing behaviors in order to determine which of the patient demographic data points for a given medical service provider fail to track with the corresponding median values.
9. The method according to embodiments 1-8, wherein the step of the computing system determining whether any practice demographic data points had an influence on the abnormal prescribing behaviors for the associated medical service providers further comprises the steps of: the computing system calculating a median value for each practice demographic data point; and the computing system comparing the calculated median values against the corresponding values for each practice demographic data point associated with each medical service provider identified as engaging in abnormal prescribing behaviors in order to determine whether any of the practice demographic data points for a given medical service fail to track with the corresponding median values.
10. The method according to embodiments 1-9, further comprising the step of the computing system generating a report for the user outlining the patient demographic data points and/or practice demographic data points having the strongest influence on the identified abnormal prescribing behaviors for a given medical service provider.
11. The method according to embodiments 1-10, wherein the step of the computing system generating a report further comprises the step of the computing system providing an at least one recommendation on how to counteract the influential patient to demographic data points and/or practice demographic data points in order to reduce or eliminate the abnormal prescribing behaviors for a given medical service provider.
12. A non-transitory computer readable medium containing program instructions for causing an at least one computing device to perform a method of analyzing medical data to identify and address abnormal prescribing behaviors, said at least one computing device in selective communication with an at least one third-party medical records database, the method comprising the steps of: receiving and processing data from the at least one medical records database related to an at least one patient and an associated at least one medical condition, along with an at least one medical service provider tasked with treating the at least one patient; establishing an at least one patient record associated with each of the at least one patient, each patient record containing at least one of a unique patient record identifier, a patient age, a patient gender, a patient ethnicity, a patient location, a patient income, a patient education level, a patient employment status, an at least one patient condition for each medical condition the associated patient has experienced or is experiencing, an associated patient prescription for each of the at least one patient condition, and an associated at least one prescription performance indicator for each of the at least one patient prescription; establishing an at least one service provider record associated with each of the at least one medical service provider, each service provider record containing at least one of a unique service provider record identifier, a service provider location, an average income representing the average income of patients treated by the associated medical service provider, an average education level representing the average education level of patients treated by the associated medical service provider, an average hours worked representing the average hours worked by patients treated by the associated medical service provider, an average crime level representing the average crime level in the corresponding service provider location, an average age representing the average age of patients treated by the associated medical service provider, an unemployment rate representing the unemployment rate in the corresponding service provider location, a mortality rate representing the mortality rate in the corresponding service provider location, and a patient table containing links to the corresponding patient record of each patient that has been treated by the associated medical service provider; and upon a user desiring to obtain an analysis of a given medical condition: generating a model of expected prescribing behaviors for said medical condition, organized by an at least one pharmaceutical product being prescribed for treating said medical condition, based on existing and generally accepted clinical guidelines; accessing data contained in the at least one service provider record related to said medical condition; stratifying the associated to medical service providers into a plurality of groups based on at least one of the respective patient demographic data points of the associated medical service providers and the prescription performance indicator of each associated patient that has been treated, or is being treated, by each of the medical service providers; generating a model of average prescribing behaviors for said medical condition, organized by the at least one pharmaceutical product being prescribed for treating said medical condition, for each of the stratified groups of medical service providers; upon determining that a given stratified group does not approximate the model of expected prescribing behaviors for said medical condition, identifying said stratified group as containing abnormal prescribing behaviors; and for any stratified groups identified as containing abnormal prescribing behaviors: determining which of the associated at least one patient demographic data point had the strongest influence on the abnormal prescribing behaviors for the associated medical service providers; and determining whether any practice demographic data points had an influence on the abnormal prescribing behaviors for the associated medical service providers.
13. The method according to embodiment 12, further comprising the steps of: accessing each of the at least one third-party medical records database and retrieving each of the entries contained within said medical records database; and for each entry retrieved from said medical records database: normalizing any text contained in said entry; processing said entry to identify any medical terms contained therewithin; for each medical term identified, examining any words surrounding said medical term to determine the presence or absence of negative modifiers that would affect whether said entry deals with the presence or absence of said medical term; and storing the data contained in said entry in at least one of a patient record and service provider record.
14. The method according to embodiments 12-13, further comprising the step of, for each medical term identified, linking said medical term with an at least one code associated with a standard medical encoding system.
15. The method according to embodiments 12-14, wherein the step of stratifying the associated medical service providers into a plurality of groups further comprises the step of weighting the associated at least one patient demographic data point based on the relative strength of said patient demographic data point's potential influence on prescribing behaviors.
16. The method according to embodiments 12-15, wherein the step of weighting the associated at least one patient demographic data point further comprises the steps of: assigning a relatively larger numerical weight to patient demographic data points related to the medical condition being analyzed than the numerical weight assigned to general patient demographic data points; and assigning a relatively larger numerical weight to the at least one prescription performance indicator associated with each of the at least one patient record having the medical condition than the numerical weight assigned to patient demographic data points related to the medical condition being analyzed.
17. The method according to embodiments 12-16, wherein the step of determining that a given stratified group does not approximate the model of expected prescribing behaviors for said medical condition, further comprises the steps of: totaling an annual prescribing amount of each pharmaceutical product being prescribed for treating said medical condition; and comparing the prescribing behavior of a given medical service provider to the average in the associated stratified group in which said medical service provider is categorized.
18. The method according to embodiments 12-17, wherein the step of determining which of the associated at least one patient demographic data point had the strongest influence on the abnormal prescribing behaviors for the associated medical service providers further comprises the steps of: calculating a median value for each patient demographic data point that was used to generate the model of average prescribing behaviors for said medical condition for each of the stratified groups; and comparing the calculated median values against the corresponding values for each patient demographic data point associated with each medical service provider identified as engaging in abnormal prescribing behaviors in order to determine which of the patient demographic data points for a given medical service provider fail to track with the corresponding median values.
19. The method according to embodiments 12-18, wherein the step of determining whether any practice demographic data points had an influence on the abnormal prescribing behaviors for the associated medical service providers further comprises the steps of: calculating a median value for each practice demographic data point; and comparing the calculated median values against the corresponding values for each practice demographic data point associated with each medical service provider identified as engaging in abnormal prescribing behaviors in order to determine whether any of the practice demographic data points for a given medical service fail to track with the corresponding median values.
20. The method according to embodiments 12-19, further comprising the step of generating a report for the user outlining the patient demographic data points and/or practice demographic data points having the strongest influence on the identified abnormal prescribing behaviors for a given medical service provider.
21. The method according to embodiments 12-20, wherein the step of generating to a report further comprises the step of the computing system providing an at least one recommendation on how to counteract the influential patient demographic data points and/or practice demographic data points in order to reduce or eliminate the abnormal prescribing behaviors for a given medical service provider.
22. A medical data analysis system for analyzing medical data to identify and address abnormal prescribing behaviors, the system comprising: an at least one computing device in selective communication with an at least one third-party medical records database, the computing device configured for receiving and processing data related to an at least one patient and an associated at least one medical condition, along with an at least one medical service provider tasked with treating the at least one patient; wherein, the at least one computing device is configured for: establishing an at least one patient record associated with each of the at least one patient, each patient record containing at least one of a unique patient record identifier, a patient age, a patient gender, a patient ethnicity, a patient location, a patient income, a patient education level, a patient employment status, an at least one patient condition for each medical condition the associated patient has experienced or is experiencing, an associated patient prescription for each of the at least one patient condition, and an associated at least one prescription performance indicator for each of the at least one patient prescription; establishing an at least one service provider record associated with each of the at least one medical service provider, each service provider record containing at least one of a unique service provider record identifier, a service provider location, an average income representing the average income of patients treated by the associated medical service provider, an average education level representing the average education level of patients treated by the associated medical service provider, an average hours worked representing the average hours worked by patients treated by the associated medical service provider, an average crime level representing the average crime level in the corresponding service provider location, an average age representing the average age of patients treated by the associated medical service provider, an unemployment rate representing the unemployment rate in the corresponding service provider location, a mortality rate representing the mortality rate in the corresponding service provider location, and a patient table containing links to the corresponding patient record of each patient that has been treated by the associated medical service provider; and upon a user desiring to obtain an analysis of a given medical condition: generating a model of expected prescribing behaviors for said medical condition, organized by an at least one pharmaceutical product being prescribed for treating said medical condition, based on existing and generally accepted clinical guidelines; accessing data contained in the at least one service provider record related to said medical condition; stratifying the associated medical service providers into a plurality of groups based on at least one of the respective patient demographic data points of the associated medical service providers and the prescription performance indicator of each associated patient that has been treated, or is being treated, by each of the medical service providers; generating a model of average prescribing behaviors for said medical condition, organized by the at least one pharmaceutical product being prescribed for treating said medical condition, for each of the stratified groups of medical service providers; upon determining that a given stratified group does not approximate the model of expected prescribing behaviors for said medical condition, identifying said stratified group as containing abnormal prescribing behaviors; and for any stratified groups identified as containing abnormal prescribing behaviors: determining which of the associated at least one patient demographic data point had the strongest influence on the abnormal prescribing behaviors for the associated medical service providers; and determining whether any practice demographic data points had an influence on the abnormal prescribing behaviors for the associated medical service providers.
23. The medical data analysis system according to embodiment 22, further comprising an at least one database in communication with the at least one computing device and configured for selectively storing said data related to the at least one patient, medical condition and medical service provider.
24. The medical data analysis system according to embodiments 22-23, wherein the at least one computing device is further configured for: accessing each of the at least one third-party medical records database and retrieving each of the entries contained within said medical records database; and for each entry retrieved from said medical records database: normalizing any text contained in said entry; processing said entry to identify any medical terms contained therewithin; for each medical term identified, examining any words surrounding said medical term to determine the presence or absence of negative modifiers that would affect whether said entry deals with the presence or absence of said medical term; and storing the data contained in said entry in at least one of a patient record and service provider record.
25. The medical data analysis system according to embodiments 22-24, wherein for each medical term identified, the at least one computing device is further configured for linking said medical term with an at least one code associated with a standard medical encoding system.
26. The medical data analysis system according to embodiments 22-25, wherein while stratifying the associated medical service providers into a plurality of groups, the at least one computing device is further configured for weighting the associated at least one patient demographic data point based on the relative strength of said patient demographic data point's potential influence on prescribing behaviors.
27. The medical data analysis system according to embodiments 22-26, wherein while weighting the associated at least one patient demographic data point, the at least one computing device is further configured for: assigning a relatively larger numerical weight to patient demographic data points related to the medical condition being analyzed than the numerical weight assigned to general patient demographic data points; and assigning a relatively larger numerical weight to the at least one prescription performance indicator associated with each of the at least one patient record having the medical condition than the numerical weight assigned to patient demographic data points related to the medical condition being analyzed.
28. The medical data analysis system according to embodiments 22-27, wherein while determining that a given stratified group does not approximate the model of expected prescribing behaviors for said medical condition, the at least one computing device is further configured for: totaling an annual prescribing amount of each pharmaceutical product being prescribed for treating said medical condition; and comparing the prescribing behavior of a given medical service provider to the average in the associated stratified group in which said medical service provider is categorized.
29. The medical data analysis system according to embodiments 22-28, wherein while determining which of the associated at least one patient demographic data point had the strongest influence on the abnormal prescribing behaviors for the associated medical service providers, the at least one computing device is further configured for: calculating a median value for each patient demographic data point that was used to generate the model of average prescribing behaviors for said medical condition for each of the stratified groups; and comparing the calculated median values against the corresponding values for each patient demographic data point associated with each medical service provider identified as engaging in abnormal prescribing behaviors in order to determine which of the patient demographic data points for a given medical service provider fail to track with the corresponding median values.
30. The medical data analysis system according to embodiments 22-29, wherein while determining whether any practice demographic data points had an influence on the abnormal prescribing behaviors for the associated medical service providers, the at least one computing device is further configured for: calculating a median value for each practice demographic data point; and comparing the calculated median values against the corresponding values for each practice demographic data point associated with each medical service provider identified as engaging in abnormal prescribing behaviors in order to determine whether any of the practice demographic data points for a given medical service fail to track with the corresponding median values.
31. The medical data analysis system according to embodiments 22-30, wherein the at least one computing device is further configured for generating a report for the user outlining the patient demographic data points and/or practice demographic data points having the strongest influence on the identified abnormal prescribing behaviors for a given medical service provider.
32. The medical data analysis system according to embodiments 22-31, wherein while generating a report, the at least one computing device is further configured for providing an at least one recommendation on how to counteract the influential patient demographic data points and/or practice demographic data points in order to reduce or eliminate the abnormal prescribing behaviors for a given medical service provider.

In closing, regarding the exemplary embodiments of the present invention as shown and described herein, it will be appreciated that a medical data analysis system and associated methods are disclosed and configured for automatically and dynamically collecting, harmonizing and analyzing medical data to identify and address abnormal prescribing behaviors. Because the principles of the invention may be practiced in a number of configurations beyond those shown and described, it is to be understood that the invention is not in any way limited by the exemplary embodiments, but is generally directed to a medical data analysis system and is able to take numerous forms to do so without departing from the spirit and scope of the invention.

Certain embodiments of the present invention are described herein, including the best mode known to the inventor(s) for carrying out the invention. Of course, variations on these described embodiments will become apparent to those of ordinary skill in the art upon reading the foregoing description. The inventor(s) expect skilled artisans to employ such variations as appropriate, and the inventor(s) intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein. Similarly, as used herein, unless indicated to the contrary, the term “substantially” is a term of degree intended to indicate an approximation of the characteristic, item, quantity, parameter, property, or term so qualified, encompassing a range that can be understood and construed by those of ordinary skill in the art.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (along with equivalent open-ended transitional phrases thereof such as “including,” “containing” and “having”) encompasses all the expressly recited elements, limitations, steps and/or features alone or in combination with un-recited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amendment for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim, whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (along with equivalent open-ended transitional phrases thereof) includes within its meaning, as a limiting case, claimed subject matter specified by the closed-ended transitional phrases “consisting of” or “consisting essentially of.” As such, embodiments described herein or so claimed with the phrase “comprising” are expressly or inherently unambiguously described, enabled and supported herein for the phrases “consisting essentially of” and “consisting of.”

Any claims intended to be treated under 35 U.S.C. § 112(f) will begin with the words “means for,” but use of the term “for” in any other context is not intended to invoke treatment under 35 U.S.C. § 112(f). Accordingly, Applicant reserves the right to pursue additional claims after filing this application, in either this application or in a continuing application.

It should be understood that the logic code, programs, modules, processes, methods, and the order in which the respective elements of each method are performed are purely exemplary. Depending on the implementation, they may be performed in any order or in parallel, unless indicated otherwise in the present disclosure. Further, the logic code is not related, or limited to any particular programming language, and may comprise one or more modules that execute on one or more processors in a distributed, non-distributed, or multiprocessing environment. Additionally, the various illustrative logical blocks, modules, methods, and algorithm processes and sequences described in connection with the embodiments disclosed herein can be implemented as electronic hardware, computer software, or combinations of both. To clearly illustrate this interchangeability of hardware and software, various illustrative components, blocks, modules, and process actions have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of this document.

The phrase “non-transitory,” in addition to having its ordinary meaning, as used in this document means “enduring or long-lived”. The phrase “non-transitory computer readable medium,” in addition to having its ordinary meaning, includes any and all computer readable mediums, with the sole exception of a transitory, propagating signal. This includes, by way of example and not limitation, non-transitory computer-readable mediums such as register memory, processor cache and random-access memory (“RAM”).

The methods as described above may be used in the fabrication of integrated circuit chips. The resulting integrated circuit chips can be distributed by the fabricator in raw wafer form (that is, as a single wafer that has multiple unpackaged chips), as a bare die, or in a packaged form. In the latter case, the chip is mounted in a single chip package (such as a plastic carrier, with leads that are affixed to a motherboard or other higher level carrier) or in a multi-chip package (such as a ceramic carrier that has either or both surface interconnections or buried interconnections). In any case, the chip is then integrated with other chips, discrete circuit elements, and/or other signal processing devices as part of either (a) an intermediate product, such as a motherboard, or (b) an end product. The end product can be any product that includes integrated circuit chips, ranging from toys and other low-end applications to advanced computer products having a display, a keyboard or other input device, and a central processor.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

While aspects of the invention have been described with reference to at least one exemplary embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

What is claimed is:

1. A method for analyzing medical data to identify and address abnormal prescribing behaviors, the method comprising the steps of:
implementing a central computing system in selective communication with an at least one third-party medical records database, the computing system configured for receiving and processing data related to an at least one patient and an associated at least one medical condition, along with an at least one medical service provider tasked with treating the at least one patient;
the computing system establishing an at least one patient record associated with each of the at least one patient, each patient record containing at least one of a unique patient record identifier, a patient age, a patient gender, a patient ethnicity, a patient location, a patient income, a patient education level, a patient employment status, an at least one patient condition for each medical condition the associated patient has experienced or is experiencing, an associated patient prescription for each of the at least one patient condition, and an associated at least one prescription performance indicator for each of the at least one patient prescription;
the computing system establishing an at least one service provider record associated with each of the at least one medical service provider, each service provider record containing at least one of a unique service provider record identifier, a service provider location, an average income representing the average income of patients treated by the associated medical service provider, an average education level representing the average education level of patients treated by the associated medical service provider, an average hours worked representing the average hours worked by patients treated by the associated medical service provider, an average crime level representing the average crime level in the corresponding service provider location, an average age representing the average age of patients treated by the associated medical service provider, an unemployment rate representing the unemployment rate in the corresponding service provider location, a mortality rate representing the mortality rate in the corresponding service provider location, and a patient table containing links to the corresponding patient record of each patient that has been treated by the associated medical service provider; and
upon a user desiring to obtain an analysis of a given medical condition:
the computing system generating a model of expected prescribing behaviors for said medical condition, organized by an at least one pharmaceutical product being prescribed for treating said medical condition, based on existing and generally accepted clinical guidelines;
the computing system accessing data contained in the at least one service provider record related to said medical condition;
the computing system stratifying the associated medical service providers into a plurality of groups based on at least one of the respective patient demographic data points of the associated medical service providers and the prescription performance indicator of each associated patient that has been treated, or is being treated, by each of the medical service providers;
the computing system generating a model of average prescribing behaviors for said medical condition, organized by the at least one pharmaceutical product being prescribed for treating said medical condition, for each of the stratified groups of medical service providers;
upon the computing system determining that a given stratified group does not approximate the model of expected prescribing behaviors for said medical condition, the computing system identifying said stratified group as containing abnormal prescribing behaviors; and
for any stratified groups identified by the computing system as containing abnormal prescribing behaviors:
the computing system determining which of the associated at least one patient demographic data point had the strongest influence on the abnormal prescribing behaviors for the associated medical service providers; and
the computing system determining whether any practice demographic data points had an influence on the abnormal prescribing behaviors for the associated medical service providers.

2. The method of claim 1, wherein the step of the computing system stratifying the associated medical service providers into a plurality of groups further comprises the step of the computing system weighting the associated at least one patient demographic data point based on the relative strength of said patient demographic data point's potential influence on prescribing behaviors.

3. The method of claim 2, wherein the step of the computing system weighting the associated at least one patient demographic data point further comprises the steps of:
the computing system assigning a relatively larger numerical weight to patient demographic data points related to the medical condition being analyzed than the numerical weight assigned to general patient demographic data points; and
the computing system assigning a relatively larger numerical weight to the at least one prescription performance indicator associated with each of the at least one patient record having the medical condition than the numerical weight assigned to patient demographic data points related to the medical condition being analyzed.

4. The method of claim 1, wherein the step of the computing system determining that a given stratified group does not approximate the model of expected prescribing behaviors for said medical condition, further comprises the steps of:
the computing system totaling an annual prescribing amount of each pharmaceutical product being prescribed for treating said medical condition; and
to the computing system comparing the prescribing behavior of a given medical service provider to the average in the associated stratified group in which said medical service provider is categorized.

5. The method of claim 1, wherein the step of the computing system determining which of the associated at least one patient demographic data point had the strongest influence on the abnormal prescribing behaviors for the associated medical service providers further comprises the steps of:
the computing system calculating a median value for each patient demographic data point that was used to generate the model of average prescribing behaviors for said medical condition for each of the stratified groups; and
the computing system comparing the calculated median values against the corresponding values for each patient demographic data point associated with each medical service provider identified as engaging in abnormal prescribing behaviors in order to determine which of the patient demographic data points for a given medical service provider fail to track with the corresponding median values.

6. The method of claim 1, wherein the step of the computing system determining whether any practice demographic data points had an influence on the abnormal prescribing behaviors for the associated medical service providers further comprises the steps of:
the computing system calculating a median value for each practice demographic data point; and
the computing system comparing the calculated median values against the corresponding values for each practice demographic data point associated with each medical service provider identified as engaging in abnormal prescribing behaviors in order to determine whether any of the practice demographic data points for a given medical service fail to track with the corresponding median values.

7. The method of claim 1, further comprising the step of the computing system generating a report for the user outlining the patient demographic data points and/or practice demographic data points having the strongest influence on the identified abnormal prescribing behaviors for a given medical service provider.

8. The method of claim 7, wherein the step of the computing system generating a report further comprises the step of the computing system providing an at least one recommendation on how to counteract the influential patient demographic data points and/or practice demographic data points in order to reduce or eliminate the abnormal prescribing behaviors for a given medical service provider.

9. A non-transitory computer readable medium containing program instructions for causing an at least one computing device to perform a method of analyzing medical data to identify and address abnormal prescribing behaviors, said at least one computing device in selective communication with an at least one third-party medical records database, the method comprising the steps of:
receiving and processing data from the at least one medical records database related to an at least one patient and an associated at least one medical condition, along with an at least one medical service provider tasked with treating the at least one patient;
establishing an at least one patient record associated with each of the at least one patient, each patient record containing at least one of a unique patient record identifier, a patient age, a patient gender, a patient ethnicity, a patient location, a patient income, a patient education level, a patient employment status, an at least one patient condition for each medical condition the associated patient has experienced or is experiencing, an associated patient prescription for each of the at least one patient condition, and an associated at least one prescription performance indicator for each of the at least one patient prescription;
establishing an at least one service provider record associated with each of the at least one medical service provider, each service provider record containing at least one of a unique service provider record identifier, a service provider location, an average income representing the average income of patients treated by the associated medical service provider, an average education level representing the average education level of patients treated by the associated medical service provider, an average hours worked representing the average hours worked by patients treated by the associated medical service provider, an average crime level representing the average crime level in the corresponding service provider location, an average age representing the average age of patients treated by the associated medical service provider, an unemployment rate representing the unemployment rate in the corresponding service provider location, a mortality rate representing the mortality rate in the corresponding service provider location, and a patient table containing links to the corresponding patient record of each patient that has been treated by the associated medical service provider; and
upon a user desiring to obtain an analysis of a given medical condition:
generating a model of expected prescribing behaviors for said medical condition, organized by an at least one pharmaceutical product being prescribed for treating said medical condition, based on existing and generally accepted clinical guidelines;
accessing data contained in the at least one service provider record related to said medical condition;
stratifying the associated medical service providers into a plurality of groups based on at least one of the respective patient demographic data points of the associated medical service providers and the prescription performance indicator of each associated patient that has been treated, or is being treated, by each of the medical service providers;
generating a model of average prescribing behaviors for said medical condition, organized by the at least one pharmaceutical product being prescribed for treating said medical condition, for each of the stratified groups of medical service providers;
upon determining that a given stratified group does not approximate the model of expected prescribing behaviors for said medical condition, identifying said stratified group as containing abnormal prescribing behaviors; and
for any stratified groups identified as containing abnormal prescribing behaviors:
determining which of the associated at least one patient demographic data point had the strongest influence on the abnormal prescribing behaviors for the associated medical service providers; and
determining whether any practice demographic data points had an influence on the abnormal prescribing behaviors for the associated medical service providers.

10. The method of claim 9, wherein the step of stratifying the associated medical service providers into a plurality of groups further comprises the step of weighting the associated at least one patient demographic data point based on the relative strength of said patient demographic data point's potential influence on prescribing behaviors.

11. The method of claim 10, wherein the step of weighting the associated at least one patient demographic data point further comprises the steps of:
assigning a relatively larger numerical weight to patient demographic data points related to the medical condition being analyzed than the numerical weight assigned to general patient demographic data points; and
assigning a relatively larger numerical weight to the at least one prescription performance indicator associated with each of the at least one patient record having the medical condition than the numerical weight assigned to patient demographic data points related to the medical condition being analyzed.

12. The method of claim 9, wherein the step of determining that a given stratified group does not approximate the model of expected prescribing behaviors for said medical condition, further comprises the steps of:
totaling an annual prescribing amount of each pharmaceutical product being prescribed for treating said medical condition; and
comparing the prescribing behavior of a given medical service provider to the average in the associated stratified group in which said medical service provider is categorized.

13. The method of claim 9, wherein the step of determining which of the associated at least one patient demographic data point had the strongest influence on the abnormal prescribing behaviors for the associated medical service providers further comprises the steps of:
calculating a median value for each patient demographic data point that was used to generate the model of average prescribing behaviors for said medical condition for each of the stratified groups; and
comparing the calculated median values against the corresponding values for each patient demographic data point associated with each medical service provider identified as engaging in abnormal prescribing behaviors in order to determine which of the patient demographic data points for a given medical service provider fail to track with the corresponding median values.

14. The method of claim 9, wherein the step of determining whether any practice demographic data points had an influence on the abnormal prescribing behaviors for the associated medical service providers further comprises the steps of:
calculating a median value for each practice demographic data point; and
comparing the calculated median values against the corresponding values for each practice demographic data point associated with each medical service provider identified as engaging in abnormal prescribing behaviors in order to determine whether any of the practice demographic data points for a given medical service fail to track with the corresponding median values.

15. A medical data analysis system for analyzing medical data to identify and address abnormal prescribing behaviors, the system comprising:
an at least one computing device in selective communication with an at least one third-party medical records database, the computing device configured for receiving and processing data related to an at least one patient and an associated at least one medical condition, along with an at least one medical service provider tasked with treating the at least one patient;
wherein, the at least one computing device is configured for:
establishing an at least one patient record associated with each of the at least one patient, each patient record containing at least one of a unique patient record identifier, a patient age, a patient gender, a patient ethnicity, a patient location, a patient income, a patient education level, a patient employment status, an at least one patient condition for each medical condition the associated patient has experienced or is experiencing, an associated patient prescription for each of the at least one patient condition, and an associated at least one prescription performance indicator for each of the at least one patient prescription;
establishing an at least one service provider record associated with each of the at least one medical service provider, each service provider record containing at least one of a unique service provider record identifier, a service provider location, an average income representing the average income of patients treated by the associated medical service provider, an average education level representing the average education level of patients treated by the associated medical service provider, an average hours worked representing the average hours worked by patients treated by the associated medical service provider, an average crime level representing the average crime level in the corresponding service provider location, an average age representing the average age of patients treated by the associated medical service provider, an unemployment rate representing the unemployment rate in the corresponding service provider location, a mortality rate representing the mortality rate in the corresponding service provider location, and a patient table containing links to the corresponding patient record of each patient that has been treated by the associated medical service provider; and
upon a user desiring to obtain an analysis of a given medical condition:
generating a model of expected prescribing behaviors for said medical condition, organized by an at least one pharmaceutical product being prescribed for treating said medical condition, based on existing and generally accepted clinical guidelines;
accessing data contained in the at least one service provider record related to said medical condition;
stratifying the associated medical service providers into a plurality of groups based on at least one of the respective patient demographic data points of the associated medical service providers and the prescription performance indicator of each associated patient that has been treated, or is being treated, by each of the medical service providers;
generating a model of average prescribing behaviors for said medical condition, organized by the at least one pharmaceutical product being prescribed for treating said medical condition, for each of the stratified groups of medical service providers;
upon determining that a given stratified group does not approximate the model of expected prescribing behaviors for said medical condition, identifying said stratified group as containing abnormal prescribing behaviors; and
for any stratified groups identified as containing abnormal prescribing behaviors:
determining which of the associated at least one patient demographic data point had the strongest influence on the abnormal prescribing behaviors for the associated medical service providers; and
determining whether any practice demographic data points had an influence on the abnormal prescribing behaviors for the associated medical service providers.

16. The medical data analysis system of claim 15, wherein while stratifying the associated medical service providers into a plurality of groups, the at least one computing device is further configured for weighting the associated at least one patient demographic data point based on the relative strength of said patient demographic data point's potential influence on prescribing behaviors.

17. The medical data analysis system of claim 16, wherein while weighting the associated at least one patient demographic data point, the at least one computing device is further configured for:
assigning a relatively larger numerical weight to patient demographic data points related to the medical condition being analyzed than the numerical weight assigned to general patient demographic data points; and
assigning a relatively larger numerical weight to the at least one prescription performance indicator associated with each of the at least one patient record having the medical condition than the numerical weight assigned to patient demographic data points related to the medical condition being analyzed.

18. The medical data analysis system of claim 15, wherein while determining that a given stratified group does not approximate the model of expected prescribing behaviors for said medical condition, the at least one computing device is further configured for:
totaling an annual prescribing amount of each pharmaceutical product being prescribed for treating said medical condition; and
comparing the prescribing behavior of a given medical service provider to the average in the associated stratified group in which said medical service provider is categorized.

19. The medical data analysis system of claim 15, wherein while determining which of the associated at least one patient demographic data point had the strongest influence on the abnormal prescribing behaviors for the associated medical service providers, the at least one computing device is further configured for:
calculating a median value for each patient demographic data point that was used to generate the model of average prescribing behaviors for said medical condition for each of the stratified groups; and
comparing the calculated median values against the corresponding values for each patient demographic data point associated with each medical service provider identified as engaging in abnormal prescribing behaviors in order to determine which of the patient demographic data points for a given medical service provider fail to track with the corresponding median values.

20. The medical data analysis system of claim 15, wherein while determining whether any practice demographic data points had an influence on the abnormal prescribing behaviors for the associated medical service providers, the at least one computing device is further configured for:
calculating a median value for each practice demographic data point; and
comparing the calculated median values against the corresponding values for each practice demographic data point associated with each medical service provider identified as engaging in abnormal prescribing behaviors in order to determine whether any of the practice demographic data points for a given medical service fail to track with the corresponding median values.
